SEQUENCE LISTING (14 sequences)

SEQ ID NO: 1 - protein, 117 aa, Artificial Sequence
  Synthetic
QVTLKESGPGILKPSQTLSLTCSFSGFSLSTSGLAVGWIRQPSGKGLEWLSHIWWNDDKYYNPSLKNQLTISKDTSRNQVFLKIISVDTADTATYYCSAYFGGKNYWGQGTALTVSS

SEQ ID NO: 2 - protein, 113 aa, Artificial Sequence
  Synthetic
DIVMTQSPSSLTVTAGEKVTMSCKSSQSLLNSGNQKNYLTWYQQKPGQPPKLLVYWASTRESGVPDRFTGSGSGTDFTLTISTVQAEDLAVYYCQNDYSYPLTFGAGTKLELK

SEQ ID NO: 3 - protein, 114 aa, Artificial Sequence
  Synthetic
DVQLVESGGGLVQPGGSRKLSCAASGFTFSRFGMNWVRQAPEKGLEWVAYISSGSGDIYYADTVKGRFTISRDNPKNTLFLQMTSLRSEDTAMYYCAVTWFAYWGQGTLVTVSA

SEQ ID NO: 4 - protein, 109 aa, Artificial Sequence
  Synthetic
QAVVTQEPALTTSPGETVTLTCRSSTGAVTTSNYANWVQEKPDHLFTGLIGGTNNRAPGVPARFSGSLIGDKAALTITGAQTEDEAIYFCVLWYSNHFIFGSGTKVTVL

SEQ ID NO: 5 - protein, 124 aa, Artificial Sequence
  Synthetic
QIQLVQSGPELKKAGETVKISCKASGYTFAAYSMHWVKQAPGKGLKWMGWINTETGEPTYADDFKGRFAFSLETSASTAYLQINNLKNEDTATYFCTRDDYDGGRFSYWYFAVWGAGTTVTVSS

SEQ ID NO: 6 - protein, 106 aa, Artificial Sequence
  Synthetic
DIQMTQSPSSLSASLGGKVTITCKASQDINKYIAWYQHRPGKGPRLLIRYTSTLQPGIPSRFSGSGSGRDYSFSINNLEPEDIATYYCLQYDNLLTFGGGTKLEIK

SEQ ID NO: 7 - protein, 120 aa, Artificial Sequence
  Synthetic
QIQLVQSGPELKKPGETVKISCKASGYTFTDYSMHWVKQAPGKGLKWMGWINTETGEPTYADDFKGRFAFSLETSASTAYLQINNLKNDDTATYFCARSFYRYDWYFDVWGAGTTVTVSS

SEQ ID NO: 8 - protein, 106 aa, Artificial Sequence
  Synthetic
QIVLTQSPAIMSASPGEKVTITCSASSSVSFMHWFQQKPGTSPKLWIYSTSNLASGVPARFSGSGSGTSYSLTISRMEAEDAATYYCQQRTSFPPTFGGGTNLEIK

SEQ ID NO: 9 - protein, 119 aa, Artificial Sequence
  Synthetic
QVTLKESGPGILQPSQTLSLTCSFSGFSLSTSNVGVGWIRQPSGKGLEWLLHILWNDGKYYNPALKSRLTISKDTSTNQVFLKIADVDTADSATYYCARLRRDYGMDYWGQGTSVTVSS

SEQ ID NO: 10 - protein, 113 aa, Artificial Sequence
  Synthetic
DIVMSQSPSSLAVSVGEKVTMSCKSSQSLLYSYNQKNYLAWYQQKPGQSPKLLIYWASTRESGVPDRFTGSGSGTDFTLTISSVKAEDLAVYYCQQYYTYPYAFGGGTKLEIK

SEQ ID NO: 11 - protein, 119 aa, Artificial Sequence
  Synthetic
QVTLKESGPGILQPSQTLSLTCSFSGFSLSTSNMGVAWIRQPLGKGLEWLLYILWNDTKYYNPALKSRLSISKDTYNNQVFLKIVNVDTADTATYYCARIRRDYALDYWGQGTSVTVSS

SEQ ID NO: 12 - protein, 113 aa, Artificial Sequence
  Synthetic
DIVMSQSPSSLAVSVGEKVTMNCKSSQSLLYSNNQKNYLAWYQQKPGQSPKLLIYWASTRESGVPDRFTGSGSGTDFTLTISSVKAEDLAVYYCQQYYIYPLAFGGGTKLEIK

SEQ ID NO: 13 - protein, 116 aa, Artificial Sequence
  Synthetic
QVQLQQSGPELVKPGASVRISCKASGYTFTSFYIHWVKQRPGQGLEWIGWISPINININYNEKFKGKATLTADKSSSTVYMQLSSLTSEDSAVYFCEGTSENFDVWGAGTTVTVSS

SEQ ID NO: 14 - protein, 112 aa, Artificial Sequence
  Synthetic
DVLMTQIPLSLPVSLGDQASISCRSTQNIVHSNGNTYLEWYLQKPGQSPKLLIYKVSNRFSGVPDRFRGSGSGTEFTLKITRVEADDLGVYYCFQGSHVPFTFGAGTKLELK

(19) United States
(12) Patent Application Publication
(10) Pub. No.: US 2021/0380707 A1
(43) Pub. Date: Dec. 9, 2021

(54) ANTI-CD70 ANTIBODIES AND USES THEREOF

(71) Applicant: CRISPR Therapeutics AG, Zug (CH)

(72) Inventors: Jason Sagert, Cambridge, MA (US); Minh Thu PHAM, Cambridge, MA (US)

(21) Appl. No.: 17/338,149

(22) Filed: Jun. 3, 2021

Related U.S. Application Data
(60) Provisional application No. 63/034,590, filed on Jun. 4, 2020.

(51) Int. Cl. C07K 16/28; G01N 33/574

(52) U.S. Cl. CPC C07K 16/2875; G01N 2800/52; G01N 2333/70575; G01N 33/57492

(57) ABSTRACT

High affinity and specificity antibodies capable of binding to human CD70. Also provided herein are methods for producing such anti-CD70 antibodies and uses thereof for detecting CD70, for example, cell surface CD70.

### CROSS REFERENCE TO RELATED APPLICATIONS

This application claims the benefit of the filing date of U.S. Provisional Application No. 63/034,590, filed Jun. 4, 2020, the entire contents of which are incorporated by reference herein.

### SEQUENCE LISTING

The application contains a Sequence Listing that has been filed electronically in the form of a text file, created Jun. 2, 2021, and named “095136-0333-029US_SEQ.TXT” (15,428 bytes), the contents of which are incorporated by reference herein in their entirety.

### BACKGROUND OF INVENTION

CD70 is a type II membrane protein and ligand for the tumor necrosis factor receptor (TNFR) superfamily member CD27 with a healthy tissue expression distribution limited to activated lymphocytes and subsets of dendritic and thymic epithelial cells and in both humans and mice.

In contrast to its tightly controlled normal tissue expression, CD70 is commonly expressed at elevated levels in multiple types of cancers, including solid tumors and hematological malignancies. As such, CD70 can serve as a treatment target and a diagnostic biomarker for such cancers.

### SUMMARY OF INVENTION

The present disclosure is based, at least in part, on the development of antibodies having high specificity and providing high sensitivity to human CD70 in assays such as immunohistochemistry (IHC) assays. Such anti-CD70 antibodies showed higher staining intensity in CD70+ tumor tissue samples relative to samples of tissues adjacent to the tumor site. As such, the anti-CD70 antibodies can be used for detecting presence of CD70 or quantifying (measuring) the level of CD70 in samples with high sensitivity and specificity.

Accordingly, one aspect of the present disclosure provides an isolated antibody, which binds a human CD70 antigen (“anti-CD70 antibody). The anti-CD70 antibodies disclosed herein may bind the same epitope of the CD70 antigen as a reference antibody or competes against the reference antibody for binding to the CD70 antigen. The reference antibody can be one of 11G12B6, 11E12E8, 4E6G9, 3H11D12E7, 19H7E4, 18F8A8, and 16D7C8. In some embodiments, the anti-CD70 antibody disclosed herein binds the CD70 antigen expressed on a cell surface, e.g., binds CD70⁺ cells.

In some embodiments, the anti-CD70 antibody disclosed herein may comprise the same heavy chain complementary determining regions (CDRs) and the same light chain complementary determining regions (CDRs) as the reference antibody. In some examples, the anti-CD70 antibody comprises the same V_{H} and/or the same V_{L} as the reference antibody.

Any of the anti-CD70 antibodies disclosed herein may be a full-length antibody or an antigen-binding fragment thereof. In some embodiments, the antibody is a human antibody or a humanized antibody. In some embodiments, any of the anti-CD70 antibodies disclosed herein may be conjugated to a detectable label.

Further, provided herein is a nucleic acid or a set of nucleic acids, which collectively encodes any of the anti-CD70 antibodies disclosed herein. In some embodiments, the nucleic acid or the set of nucleic acids can be a vector or a set of vectors, for example, expression vectors. Also within the scope of the present disclosure are host cells (e.g., mammalian cells) comprising the nucleic acid or the set of nucleic acids coding for the anti-CD70 antibodies disclosed herein.

In another aspect, the present disclosure provides a method for detecting or quantifying CD70 in a sample, the method comprising: (i) contacting any of the anti-CD70 antibodies disclosed herein of with a sample suspected of containing CD70 (e.g., cell surface CD70), and (ii) detecting binding of the antibody to the CD70.

In yet another aspect, the present disclosure provides a method for identifying a human patient suitable for an anti-CD70 therapy, the method comprising: (i) providing a biological sample from a human patient in need thereof; (ii) contacting any of the anti-CD70 antibodies with the biological sample; (iii) detecting binding of the antibody to CD70 in the biological sample, if any; (iv) determining presence or measuring the level of CD70 in the biological sample based on result of step (iii); and (v) identifying the human patient as suitable for an anti-CD70 therapy based on the presence or the level of CD70 determined in step (iv). In some instances, the presence or the level of CD70⁺ cells is determined in step (iv). Any of the human patient identified as suitable for an anti-CD70 therapy may be subject to a treatment involving an anti-CD70 agent, such as an anti-CD70 antibody or T cells expression an anti-CD70 chimeric antigen receptor (CAR).

In any of the methods disclosed herein, the sample may be a biological sample. Examples include a tissue sample or a blood sample. In some instances, the biological sample may be a formalin-fixed paraffin-embedded tissue sample. In some embodiments, the biological sample is derived from a human patient having or suspected of having a disease involving CD70+ cells, for example, a solid tumor or a hematological malignancy (e.g., T cell malignancy or B cell malignancy). In some instances, the solid tumor or the hematological malignancy is refractory or relapsed. Exemplary solid tumors include, but are not limited to, pancreatic cancer, gastric cancer, ovarian cancer, cervical cancer, breast cancer, renal cancer, thyroid cancer, nasopharyngeal cancer, non-small cell lung (NSCLC), glioblastoma, and melanoma. Exemplary hematological malignancies include, but are not limited to, peripheral T cell lymphoma (PTCL), anaplastic large cell lymphoma (ALCL), Sezary syndrome (SS), non-smoldering acute adult T cell leukemia or lymphoma (ATLL), angioimmunoblastic T cell lymphoma (AITL), and diffuse large B cell lymphoma (DLBCL).

In some examples, the biological sample comprises a tumor tissue sample, a sample of a non-tumor tissue sample (e.g., a sample of a tissue adjacent to a tumor site), or a combination thereof. When the CD70 level in the tumor tissue sample is higher than the CD70 level in a non-tumor tissue sample, such as in the tumor adjacent tissue sample, as determined using any of the anti-CD70 antibodies disclosed herein, the human patient may be identified as suitable for an anti-CD70 therapy. In other examples, the human patient may be identified as suitable for an anti-CD70 therapy if a tumor tissue sample or a blood sample of that patient contains CD70+ tumor cells as determined using any of the anti-CD70 antibodies disclosed herein (e.g., 11E12E8, 4E6G9, or 3H11D12E7). In other examples, the human patient may be identified as suitable for an anti-CD70 therapy if a tumor tissue sample or a blood sample of that patient contains 10% or more CD70+tumor cells as determined using any of the anti-CD70 antibodies disclosed herein (e.g., 11E12E8, 4E6G9, or 3H11D12E7).

In specific examples, any of the anti-CD70 antibodies disclosed herein (e.g., 11E12E8, 4E6G9, or 3H11D12E7) can be used in an immunohistochemistry (IHC) assay to detect presence and/or measure levels of CD70 in a biological sample, which may be obtained from a human patient (e.g., those disclosed herein). In some instances, the biological sample can be an FFPE sample.

In addition, the present disclosure features a method of producing an antibody binding to human CD70, the method comprising: (i) culturing the host cell comprising coding sequences for any of the anti-CD70 antibodies in operable linkage to a promoter under conditions allowing for expression of the antibody that binds human CD70; and (ii) harvesting the antibody thus produced from the cell culture. In some embodiments, the method further comprise (iii) purifying the antibody after step (ii).

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 provides images from immunohistochemistry (IHC) staining of renal clear cell carcinoma (RCC) tissues and tissues adjacent to the tumor (a.k.a., cancer adjacent kidney tissues) with purified monoclonal antibodies described herein. RCC tissue (RCC; top); Cancer adjacent kidney tissue (Control; bottom). RnD: a control antibody.

FIG. 2 provides images from IHC staining of renal clear cell carcinoma (RCC) tissues and tissues adjacent to the tumor (a.k.a., cancer adjacent kidney tissues) with purified monoclonal antibodies described herein under different epitope retrieval solutions. RCC tissue (RCC; top); Cancer adjacent kidney tissue (Control; bottom). Epitope retrieval solution 1 (ER1); Epitope retrieval solution 2 (ER2). RnD: a control antibody.

FIGS. 3A and 3B include diagrams showing correlation between CD70 IHC staining and mRNA expression assays by RNA-seq. FIG. 3A: formalin fixed paraffin embedded (FFPE) tumor tissue samples from RCC, pancreas, lung, and head and neck cancer patients. FIG. 3B: FFPE tumor tissue samples from RCC patients.

### DETAILED DESCRIPTION OF THE INVENTION

Provided herein are antibodies capable of binding to human CD70 (anti-CD70 antibodies) with high binding affinity and/or specificity. As shown herein, exemplary anti-CD70 antibodies provided herein (e.g., 4E6G9, 3H11D12E7 and 11E12E8) provided strong staining signals in tumor tissue samples relative to non-tumor samples such as tissue samples adjacent to the tumor sites. Further, exemplary anti-CD70 antibodies disclosed herein (e.g., 4E6G9) successfully detected the presence of CD70 in in immunohistochemistry assays, for example, using formalin-fixed paraffin-embedded (FFPE) tumor tissue samples, and detected CD70 protein expression in tumor tissue samples. The CD70 levels in tumor tissue samples detected in IHC assays using the anti-CD70 antibodies disclosed herein correlate with the CD70 mRNA levels in the same tumor tissue samples, indicating that the anti-CD70 antibodies disclosed herein can be used to measure CD70 levels in tissue samples.

Historically, it has been difficult to obtain anti-CD70 antibodies that can be used in IHC to detect presence of CD70 proteins in FFPE samples. See, e.g., Ryan et al., 2010. The anti-CD70 antibodies disclosed herein (e.g., 4E6G9, 3H11D12E7 and 11E12E8) exhibit improved function in detecting CD70 positive cancer cells in biological samples, for example, in FFPE samples. The anti-CD70 antibodies disclosed herein show more intense and more consistent plasma membrane staining, making it easier to distinguish CD70-positive samples from CD70-negative samples.

Taken together, the anti-CD70 antibodies disclosed herein (e.g., 4E6G9, 3H11D12E7 and 11E12E8) are thus superior diagnostic antibodies for detecting presence or quantifying levels of CD70 in IHC assays, for example, in samples such as blood samples or tissue samples (e.g., FFPE samples).

### I. Antibodies Binding to Human CD70

Provided herein are antibodies capable of binding to human CD70 antigen, for example, binding to the extracellular domain of the human CD70 antigen. CD70 is a type II membrane protein and ligand for the tumor necrosis factor receptor (TNFR) superfamily member CD27. The amino acid sequences for human CD70 can be found, for example, under GenBank accession no. NP_001232.1 (isoform 1) or NP_001317261.1 (isoform 2).

An antibody (interchangeably used in plural form) is an immunoglobulin molecule capable of specific binding to a target, such as a human CD70 antigen or the extracellular domain thereof, in the present application, through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of an immunoglobulin molecule that comprises an antigen recognition site of the required specificity, including glycosylation variants of antibodies, amino acid sequence variants of antibodies, and covalently modified antibodies. An antibody as disclosed herein includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAl and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

The anti-CD70 antibodies described herein may be a full-length antibody, which contains two heavy chains and two light chains, each including a variable domain and a constant domain. Alternatively, the anti-CD70 antibodies described herein can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The anti-CD70 antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the anti-CD70 antibodies described herein, e.g., antibody 11G12B6, 11E12E8, 4E6G9, 3H11D12E7, 19H7E4, 18F8A8, or 16D7C8, can be either monoclonal or polyclonal. A “monoclonal antibody” refers to a homogenous antibody population and a “polyclonal antibody” refers to a heterogeneous antibody population. These two terms do not limit the source of an antibody or the manner, in which it is made.

In some embodiments, the anti-CD70 antibodies described herein are human antibodies, which may be isolated from a human antibody library or generated in transgenic mice. For example, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the antibody library display technology, such as phage, yeast display, mammalian cell display, or mRNA display technology as known in the art can be used to produce human antibodies and antibody fragments in vitro, from immunoglobulin variable (V) domain gene repertoires from unimmunized donors.

In other embodiments, the anti-CD70 antibodies described herein may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-CD70 antibodies described herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, the anti-CD70 antibodies described herein specifically bind to the corresponding target antigen (i.e., a human CD70 or an extracellular domain thereof) or an epitope thereof. An antibody that “specifically binds” to an antigen or an epitope is a term well understood in the art. A molecule is said to exhibit “specific binding” if it reacts more frequently, more rapidly, with greater duration, with greater avidity, and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method).

In some embodiments, the anti-CD70 antibodies described herein (e.g., antibody 11G12B6, 11E12E8, 4E6G9, 3H11D12E7, 19H7E4, 18F8A8, or 16D7C8) have a suitable binding affinity for the target antigen (i.e., a human CD70 antigen or an extracellular domain thereof) or antigenic epitopes thereof. As used herein, “binding affinity” refers to the apparent association constant or K_{A}. The KA is the reciprocal of the dissociation constant (K_{D}). The antibody described herein may have a binding affinity (K_{D}) of at least 100 mM, 10 mM, 1 mM, 0.1 mM, 100 μM, 10 μM, 1 μM, 0.1 μM, 100 nM, 10 nM, 1 nM, 0.1 nM, or lower for the CD70 antigen. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the antibodies disclosed herein may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of K_{A}, since sometimes it is sufficient to obtain a quantitative measurement of affinity (e.g., determined using a method such as ELISA or FACS analysis), which is proportional to K_{A}. The quantitative measurement thus can be used for comparisons, such as determining whether a higher affinity is, e.g., 2-fold higher, so as to obtain a qualitative measurement of affinity, or to obtain an inference of affinity, e.g., by activity in a functional assay, e.g., an in vitro or in vivo assay.

The structural information (heavy chain and light chain variable domains) of exemplary anti-CD70 antibodies (11G12B6, 11E12E8, 4E6G9, 3H11D12E7, 19H7E4, 18F8A8, or 16D7C8) are provided in Table 1 below. The heavy chain CDRs and light chain CDRs (determined by the Kabat approach; see, e.g., Kabat, E. A., et al. (1991) Sequences of Proteins of Immunological Interest, Fifth Edition, U.S. Department of Health and Human Services, NIH Publication No. 91-3242, imgt.org/IMGTindex/V-QUEST.php, and ncbi.nlm.nih.gov/igblast/) are identified in boldface. See also Table 1 below.

In some embodiments, the anti-CD70 antibodies described herein bind to the same epitope in a human CD70 or an extracellular domain thereof as an exemplary antibody 11G12B6, 11E12E8, 4E6G9, 3H11D12E7, 19H7E4, 18F8A8, or 16D7C8 or compete against the exemplary antibody (a.k.a. reference antibody) for binding to the CD70 antigen. An “epitope” as used herein refers to the site on a target antigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residues, less than 2 non-overlapping amino acid residues, or only 1 non-overlapping amino acid residue) as the exemplary antibody. Whether two antibodies compete against each other for binding to the cognate antigen can be determined by a competition assay, which is well known in the art. In some examples, the anti-CD70 antibody disclosed herein binds to the same epitope as exemplary antibody 11E12E8 or competes against 11E12E8 from binding to the CD70 antigen. In some examples, the anti-CD70 antibody disclosed herein binds to the same epitope as exemplary antibody 4E6G9 or competes against 4E6G9 from binding to the CD70 antigen. In some examples, the anti-CD70 antibody disclosed herein binds to the same epitope as exemplary antibody 3H11D12E7 or competes against 3H11D12E7 from binding to the CD70 antigen.

In some examples, the anti-CD70 antibodies disclosed herein comprises the same V_{H} and/or V_{L} CDRs as the exemplary antibody 11G12B6, 11E12E8, 4E6G9, 3H11D12E7, 19H7E4, 18F8A8, or 16D7C8. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such antibodies may have the same V_{H}, the same V_{L}, or both as compared to an exemplary antibody described herein. The heavy chain and light chain CDRs of exemplary antibody 11G12B6, 11E12E8, 4E6G9, 3H11D12E7, 19H7E4, 18F8A8, or 16D7C8, determined by the Kabat approach as an example, are provided in Table 1 below. In some specific examples, the anti-CD70 antibodies disclosed herein comprises the same V_{H} and/or V_{L} CDRs as exemplary antibody 11E12E8. In some specific examples, the anti-CD70 antibodies disclosed herein comprises the same V_{H} and/or V_{L} CDRs as exemplary antibody 4E6G9. In some specific examples, the anti-CD70 antibodies disclosed herein comprises the same V_{H} and/or V_{L} CDRs as exemplary antibody 3H11D12E7.

Also within the scope of the present disclosure are functional variants of exemplary antibody 11G12B6, 11E12E8, 4E6G9, 3H11D12E7, 19H7E4, 18F8A8, or 16D7C8. In some specific examples, provided herein are functional variants of exemplary antibody 11E12E8. In some specific examples, provided herein are functional variants of exemplary antibody 4E6G9. In some specific examples, provided herein are functional variants of exemplary antibody 3H11D12E7. Such functional variants are substantially similar to the exemplary antibody, both structurally and functionally. A functional variant comprises substantially similar V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope in CD70 with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an antibody may comprise a V_{H} fragment having CDR amino acid residue variations in only the heavy chain CDR1 as compared with the V_{H} of the exemplary antibody. In some examples, the antibody may further comprise a V_{L} fragment having the same V_{L} CDR3, and optionally the same V_{L} CDR1 or V_{L} CDR2 as the exemplary antibody.

In some instances, the amino acid residue variations (e.g., in one or more of the heavy chain and light chain CDRs of antibody 11E12E8, 4E6G9, or 3H11D12E7) can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D.

In some embodiments, the anti-CD70 antibodies disclosed herein may comprise heavy chain CDRs that are at least 80% (e.g., 85%, 90%, 95%, or 98%) identical, individually or collectively, as compared with the V_{H} CDRs of the exemplary antibody 11G12B6, 11E12E8, 4E6G9, 3H11D12E7, 19H7E4, 18F8A8, or 16D7C8. In some specific examples, the anti-CD70 antibodies disclosed herein may comprise heavy chain CDRs that are at least 80% (e.g., 85%, 90%, 95%, or 98%) identical, individually or collectively, as compared with the V_{H} CDRs of the exemplary antibody 11E12E8. In other specific examples, the anti-CD70 antibodies disclosed herein may comprise heavy chain CDRs that are at least 80% (e.g., 85%, 90%, 95%, or 98%) identical, individually or collectively, as compared with the V_{H} CDRs of the exemplary antibody 4E6G9. In yet other specific examples, the anti-CD70 antibodies disclosed herein may comprise heavy chain CDRs that are at least 80% (e.g., 85%, 90%, 95%, or 98%) identical, individually or collectively, as compared with the V_{H} CDRs of the exemplary antibody 3H11D12E7.

Alternatively or in addition, the anti-CD70 antibodies disclosed herein may comprise light chain CDRs that are at least 80% (e.g., 85%, 90%, 95%, or 98%) identical, individually or collectively, as compared with the V_{L} CDRs as the exemplary antibody 11G12B6, 11E12E8, 4E6G9, 3H11D12E7, 19H7E4, 18F8A8, or 16D7C8. In some specific examples, the anti-CD70 antibodies disclosed herein may comprise light chain CDRs that are at least 80% (e.g., 85%, 90%, 95%, or 98%) identical, individually or collectively, as compared with the V_{L} CDRs as the exemplary antibody 11E12E8. In other specific examples, the anti-CD70 antibodies disclosed herein may comprise light chain CDRs that are at least 80% (e.g., 85%, 90%, 95%, or 98%) identical, individually or collectively, as compared with the V_{L} CDRs as the exemplary antibody 4E6G9. In yet other specific examples, the anti-CD70 antibodies disclosed herein may comprise light chain CDRs that are at least 80% (e.g., 85%, 90%, 95%, or 98%) identical, individually or collectively, as compared with the V_{L} CDRs as the exemplary antibody 3H11D12E7.

As used herein, “individually” means that one CDR of an antibody shares the indicated sequence identity relative to the corresponding CDR of the exemplary antibody. “Collectively” means that three V_{H} or V_{L} CDRs of an antibody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-CD70 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

TABLE 1
V_{H} and V_{L} Sequences of Exemplary Anti-CD70 Antibodies.

| Antibodies | | Sequences |
|---|---|---|
| 11G12B6 | VH | QVTLKESGPGILKPSQTLSLTCSFSGFSLSTSGLAVGWIRQPSGKGLEWLS |
| | | HIWWNDDKYYNPSLKNQLTISKDTSRNQVFLKIISVDTADTATYYCSAYFG |
| | | GKNYWGQGTALTVSS |
| | VL | DIVMTQSPSSLTVTAGEKVTMSCKSSQSLLNSGNQKNYLTWYQQKPGQPPK |
| | | LLVYWASTRESGVPDRFTGSGSGTDFTLTISTVQAEDLAVYYCQNDYSYPL |
| | | TFGAGTKLELK |
| 11E12E8 | VH | DVQLVESGGGLVQPGGSRKLSCAASGFTFSRFGMNWVRQAPEKGLEWVAYI |
| | | SSGSGDIYYADTVKGRFTISRDNPKNTLFLQMTSLRSEDTAMYYCAVTWFA |
| | | YWGQGTLVTVSA |
| | VL | QAVVTQEPALTTSPGETVTLTCRSSTGAVTTSNYANWVQEKPDHLFTGLIG |
| | | GTNNRAPGVPARFSGSLIGDKAALTITGAQTEDEAIYFCVLWYSNHFIFGS |
| | | GTKVTVL |
| 4E6G9 | VH | QIQLVQSGPELKKAGETVKISCKASGYTFAAYSMHWVKQAPGKGLKWMGWI |
| | | NTETGEPTYADDFKGRFAFSLETSASTAYLQINNLKNEDTATYFCTRDDYD |
| | | GGRFSYWYFAVWGAGTTVTVSS |
| | VL | DIQMTQSPSSLSASLGGKVTITCKASQDINKYIAWYQHRPGKGPRLLIRYT |
| | | STLQPGIPSRFSGSGSGRDYSFSINNLEPEDIATYYCLQYDNLLTFGGGTK |
| | | LEIK |
| 3H11D12E7 | VH | QIQLVQSGPELKKPGETVKISCKASGYTFTDYSMHWVKQAPGKGLKWMGWI |
| | | NTETGEPTYADDFKGRFAFSLETSASTAYLQINNLKNDDTATYFCARSFYR |
| | | YDWYFDVWGAGTTVTVSS |
| | VL | QIVLTQSPAIMSASPGEKVTITCSASSSVSFMHWFQQKPGTSPKLWIYSTS |
| | | NLASGVPARFSGSGSGTSYSLTISRMEAEDAATYYCQQRTSFPPTFGGGTN |
| | | LEIK |
| 19H7E4 | VH | QVTLKESGPGILQPSQTLSLTCSFSGFSLSTSNVGVGWIRQPSGKGLEWLL |
| | | HILWNDGKYYNPALKSRLTISKDTSTNQVFLKIADVDTADSATYYCARLRR |
| | | DYGMDYWGQGTSVTVSS |
| | VL | DIVMSQSPSSLAVSVGEKVTMSCKSSQSLLYSYNQKNYLAWYQQKPGQSPK |
| | | LLIYWASTRESGVPDRFTGSGSGTDFTLTISSVKAEDLAVYYCQQYYTYPY |
| | | AFGGGTKLEIK |
| 18F8A8 | VH | QVTLKESGPGILQPSQTLSLTCSFSGFSLSTSNMGVAWIRQPLGKGLEWLL |
| | | YILWNDTKYYNPALKSRLSISKDTYNNQVFLKIVNVDTADTATYYCARIRR |
| | | DYALDYWGQGTSVTVSS |
| | VL | DIVMSQSPSSLAVSVGEKVTMNCKSSQSLLYSNNQKNYLAWYQQKPGQSPK |
| | | LLIYWASTRESGVPDRFTGSGSGTDFTLTISSVKAEDLAVYYCQQYYIYPL |
| | | AFGGGTKLEIK |
| 16D7C8 | VH | QVQLQQSGPELVKPGASVRISCKASGYTFTSFYIHWVKQRPGQGLEWIGWI |
| | | SPINININYNEKFKGKATLTADKSSSTVYMQLSSLTSEDSAVYFCEGTSEN |
| | | FDVWGAGTTVTVSS |
| | VL | DVLMTQIPLSLPVSLGDQASISCRSTQNIVHSNGNTYLEWYLQKPGQSPKL |
| | | LIYKVSNRFSGVPDRFRGSGSGTEFTLKITRVEADDLGVYYCFQGSHVPFT |
| | | FGAGTKLELK |

### II. Preparation of Anti-CD70 Antibodies

The anti-CD70 antibodies described herein (e.g., antibody 11G12B6, 11E12E8, 4E6G9, 3H11D12E7, 19H7E4, 18F8A8, or 16D7C8) can be made by any method known in the art. See, for example, Harlow and Lane, (1998) Antibodies: A Laboratory Manual, Cold Spring Harbor Laboratory, New York.

In some embodiments, the anti-CD70 antibody may be produced by the conventional hybridoma technology. The full-length human CD70 or an extracellular fragment thereof, optionally coupled to a carrier protein such as KLH or fused to an Fc fragment, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-CD70 monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as a source of antibodies encompasses all derivatives, progeny cells of the parent hybridomas that produce monoclonal antibodies capable of binding to CD70. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma cell line) may be sequenced and the polynucleotide sequence may then be cloned into a vector for expression or propagation. The sequence encoding the antibody of interest may be maintained in the vector in a host cell and the host cell can then be expanded and frozen for future use. In an alternative, the polynucleotide sequence may be used for genetic manipulation to, e.g., humanize the antibody or to improve the affinity (affinity maturation), or other characteristics of the antibody. For example, the constant region may be engineered to more resemble human constant regions to avoid immune response if the antibody is from a non-human source and is to be used in clinical trials and treatments in humans. Alternatively, or in addition, it may be desirable to genetically manipulate the antibody sequence to obtain greater affinity and/or specificity to the target antigen. It will be apparent to one of skill in the art that one or more polynucleotide changes can be made to the antibody and still maintain its binding specificity to the target antigen.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen, in which various fragments of the CD70 protein have been replaced (swapped) with sequences from a closely related, but antigenically distinct protein. By assessing binding of the antibody to the mutant CD70 polypeptide, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some embodiments, the anti-CD70 antibodies disclosed herein can be produced using the conventional recombinant technology as exemplified below.

Nucleic acids encoding the heavy and light chain of an antibody described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters (Brown, M. et al., Cell, 49:603-612 (1987)), those using the tetracycline repressor (tetR) (Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)). Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters (M. Brown et al., Cell, 49:603-612 (1987)); Gossen and Bujard (1992); (M. Gossen et al., Natl. Acad. Sci. USA, 89:5547-5551 (1992)) combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(11):1811-1818, 1999). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an antibody described herein. The recombinant expression vector can be introduced into a suitable host cell (e.g., a dhfr-CHO cell) by a conventional method, e.g, calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody. Other types of host cells, for example, mammalian cells, bacterial cells, yeast cells, or insect cells, may also be used to produce the anti-CD70 antibodies disclosed herein.

In one example, two recombinant expression vectors are provided, one encoding the heavy chain of an antibody described herein (e.g., antibody 11G12B6, 11E12E8, 4E6G9, 3H11D12E7, 19H7E4, 18F8A8, or 16D7C8) and the other encoding the light chain of the antibody described herein (e.g., antibody 11G12B6, 11E12E8, 4E6G9, 3H11D12E7, 19H7E4, 18F8A8, or 16D7C8). Both of the two recombinant expression vectors can be introduced into a suitable host cell (e.g., dhfr-CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-CD70 antibody as described herein (e.g., antibody 11G12B6, 11E12E8, 4E6G9, 3H11D12E7, 19H7E4, 18F8A8, or 16D7C8), vectors (e.g., expression vectors) containing such, and host cells comprising the vectors are within the scope of the present disclosure.

In other embodiments, the anti-CD70 antibodies described herein can be single-chain antibody fragments (scFv). A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a human CD70 antigen or an extracellular domain thereof, which can be identified from the library following routine procedures. Positive clones can be subjected to further screening to identify those that bind CD70 antigen or a fragment thereof.

Any of the methods for producing the anti-CD70 antibodies disclosed herein and the antibodies thus produced are also within the scope of the present disclosure.

### III. Applications of Anti-CD70 Antibodies

The present disclosure also provides methods for detecting or quantifying (measuring) a human CD70 antigen in a sample using any of the anti-CD70 antibodies as described herein (e.g., antibody 11G12B6, 11E12E8, 4E6G9, 3H11D12E7, 19H7E4, 18F8A8, or 16D7C8). In some examples, any of the detecting or diagnosing methods disclosed herein use the anti-CD70 antibody 11E12E8 or a functional variant thereof as disclosed above. In some examples, any of the detecting or diagnosing methods disclosed herein use the anti-CD70 antibody 4E6G9 or a functional variant thereof as disclosed above. In some examples, any of the detecting or diagnosing methods disclosed herein use the anti-CD70 antibody 3H11D12E7 or a functional variant thereof as disclosed above.

To perform the method disclosed herein, any of the anti-CD70 antibodies can be brought in contact with a sample suspected of containing a target antigen as disclosed herein, for example, a human CD70 protein or a CD70⁺ cell. In general, the term “contacting” or “in contact” refers to an exposure of the anti-CD70 antibody disclosed herein with the sample suspected of containing the target antigen for a suitable period under suitable conditions sufficient for the formation of a complex between the anti-CD70 antibody and the target antigen in the sample, if any. In some embodiments, the contacting is performed by capillary action in which a sample is moved across a surface of the support membrane. The antibody-antigen complex thus formed, if any, can be determined via a routine approach. Detection of such an antibody-antigen complex after the incubation is indicative of the presence of the target antigen in the sample. When needed, the amount of the antibody-antigen complex can be quantified, which is indicative of the level of the target antigen in the sample.

A suitable concentration of the anti-CD70 antibody can be used in the assay methods disclosed herein, for example, about 1 μg/ml to about 10 μg/ml. In some instances, the concentration of the anti-CD70 antibody for use in the assay methods disclosed herein (e.g., an IHC assay) can be around 1 μg/ml to about 5 μg/ml (e.g., 1, 2, 3, 4, or 5 μg/ml). In other instances, the concentration of the anti-CD70 antibody for use in the assay methods disclosed herein (e.g., an IHC assay) can be around 5 μg/ml to about 10 μg/ml (e.g., 5, 6, 7, 8, 9 or 10 μg/ml).

In some examples, about 1.25 μg/ml of the anti-CD70 antibody as disclosed herein (e.g., 4E6G9 or 3H11D12E7) may be used. In other examples, about 2.5 μg/ml of the anti-CD70 antibody as disclosed herein (e.g., 4E6G9 or 3H11D12E7). Alternative, about 5 μg/ml of the anti-CD70 antibody as disclosed herein (e.g., 4E6G9 or 3H11D12E7). In other examples, about 7.5 μg/ml of the anti-CD70 antibody as disclosed herein (e.g., 4E6G9 or 3H11D12E7). In yet other examples, about 8 μg/ml of the anti-CD70 antibody as disclosed herein (e.g., 4E6G9 or 3H11D12E7). In one specific example, about 10 μg/ml of the anti-CD70 antibody as disclosed herein (e.g., 4E6G9 or 3H11D12E7).

In some embodiments, a target antigen disclosed herein such as a human CD70 antigen or a CD70⁺ cell in a sample can be detected or quantified using any of the anti-CD70 antibodies disclosed herein via an immunoassay. Examples of immunoassays include, without limitation, immunoblotting assay (e.g., Western blot), immunohistochemical analysis, flow cytometry assay, immunofluorescence assay (IF), enzyme linked immunosorbent assays (ELISAs) (e.g., sandwich ELISAs), radioimmunoassays, electrochemiluminescence-based detection assays, magnetic immunoassays, lateral flow assays, and related techniques. Additional suitable immunoassays for detecting the target antigen in a sample will be apparent to those of skill in the art.

In some examples, the anti-CD70 antibodies as described herein (e.g., antibodies comprising the same heavy chain and light chain CDRs or comprising the same V_{H} and the same V_{L}, as antibody 11G12B6, 11E12E8, 4E6G9, 3H11D12E7, 19H7E4, 18F8A8, or 16D7C8) can be conjugated to a detectable label, which can be any agent capable of releasing a detectable signal directly or indirectly. In specific examples, the antibody has the same heavy chain and light chain CDRs or comprising the same V_{H} and the same V_{L} as antibody 11E12E8. In other specific examples, the antibody has the same heavy chain and light chain CDRs or comprising the same V_{H} and the same V_{L} as antibody 4E6G9. In yet other specific examples, the antibody has the same heavy chain and light chain CDRs or comprising the same V_{H} and the same V_{L} as antibody 3H11D12E7. The presence of such a detectable signal or intensity of the signal is indicative of presence or quantity of the target antigen in the sample.

Alternatively, a secondary antibody specific to the anti-CD70 antibody or specific to the target antigen may be used in the methods disclosed herein. For example, when the anti-CD70 antibody used in the method is a full-length antibody, the secondary antibody may bind to the constant region of the anti-CD70 antibody. In other instances, the secondary antibody may bind to an epitope of the target antigen that is different from the binding epitope of the anti-CD70 antibody. Any of the secondary antibodies disclosed herein may be conjugated to a detectable label.

Any suitable detectable label known in the art can be used in the assay methods described herein. In some embodiments, a detectable label can be a label that directly releases a detectable signal. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

In some embodiments, the detectable label may be a molecule that releases a detectable signal indirectly, for example, via conversion of a reagent to a product that directly releases the detectable signal. In some examples, such a detectable label may be an enzyme (e.g., β-galactosidase, HRP or AP) capable of producing a colored product from a colorless substrate.

Any of the anti-CD70 antibodies disclosed herein can be used for detecting and/or quantifying cells (e.g., cancer cells) that express surface CD70. In some embodiments, any of the anti-CD70 antibodies disclosed herein can be used to identify patients suitable for anti-CD70 treatments (e.g., anti-CD70 antibody treatment or anti-CD70 CAR-T treatment). To perform this method, one or more biological samples can be obtained from a candidate patient, e.g., a human patient suspected of having a disorder involving CD70+ cells. Presence of CD70+ cells or the level of CD70+ cells in the biological samples can be detected using any of the anti-CD70 antibodies disclosed here, e.g., 11E12E8, 4E6G9, or 3H11D12E7, or a functional variant thereof. Presence or the level of CD70+cells thus determined can be used as an indication for selecting patients suitable for an anti-CD70 therapy.

As used herein, a “biological sample” refers to a composition that comprises tissue, e.g., organ tissue, blood, plasma or protein, from a subject. A biological sample can be an initial unprocessed sample taken from a subject or a subsequently processed sample, e.g., partially purified or preserved forms. In some embodiments, multiple (e.g., at least 2, 3, 4, 5, or more) biological samples may be collected from a subject, over time or at particular time intervals. In some examples, a biological sample may comprise a tumor tissue sample. In other examples, the biological sample may comprise non-tumor tissues. For example, the biological sample may comprise tissues adjacent to a tumor site. In some embodiment, the biological sample can be obtained from a patient having a solid tumor. For example, the biological sample may be a tissue sample (e.g., an FFPE sample) or a blood sample comprising tumor cells. The biological samples may comprise tumor cells of pancreatic cancer, gastric cancer, ovarian cancer, cervical cancer, breast cancer, renal cancer, thyroid cancer, nasopharyngeal cancer, non-small cell lung carcinoma (NSCLC), glioblastoma, lymphoma, and/or melanoma. Alternatively or in addition, the biological sample may be a tissue sample of pancreas, kidney, gastric tract, ovary, cervix, breast, ling, liver, nasopharynx, brain, bone marrow, or skin.

The terms “patient,” “subject,” or “individual” may be used interchangeably and refer to a subject who needs the analysis as described herein. In some embodiments, the subject is a human patient, which has or is suspected of having a disease associated with CD70+ cells, for example, cancer. In some examples, the human patient has or is suspected of having a solid tumor. Examples include, but are not limited to, pancreatic cancer, gastric cancer, ovarian cancer, cervical cancer, breast cancer, renal cancer, thyroid cancer, nasopharyngeal cancer, non-small cell lung carcinoma (NSCLC), glioblastoma, and melanoma. In one specific example, the patient has or is suspected of having renal cell carcinoma (RCC). In other examples, the human patient has or is suspected of having a hematological malignancy, such as a T cell malignancy or a B cell malignancy. Examples include, but are not limited to, peripheral T cell lymphoma (PTCL), anaplastic large cell lymphoma (ALCL), Sezary syndrome (SS), non-smoldering acute adult T cell leukemia or lymphoma (ATLL), angioimmunoblastic T cell lymphoma (AITL), and diffuse large B cell lymphoma (DLBCL).

A patient who has CD70+ disease cells (e.g., cancer cells) or who has an elevated level of CD70 in tumor tissues relative to normal tissues or non-tumor tissues (e.g., tissues adjacent to a tumor site) may be identified as suitable for an anti-CD70 therapy. In some embodiments, an anti-CD70 antibody disclosed herein (e.g., 11E12E8, 4E6G9, or 3H11D12E7, or a functional variant thereof) can be used in an immunohistochemistry (IHC) assay to measure the level of CD70 in a tumor tissue sample. A patient having CD70+ cells in the tumor tissue sample may be identified as suitable for an anti-CD70 therapy (e.g., an anti-CD70 CAR-T therapy). For example, a patient having CD70+ cells (e.g., at least 5%, at least 10%, at least 15%, at least 20%, at least 25% or higher) in the tumor tissue sample may be identified as suitable for an anti-CD70 therapy (e.g., an anti-CD70 CAR-T therapy). In other embodiments, an anti-CD70 antibody disclosed herein (e.g., 11E12E8, 4E6G9, or 3H11D12E7, or a functional variant thereof) can be used in a flow cytometry assay to measure the level of CD70 in a blood sample collected from a candidate cancer patient. A patient having CD70+ cells in tumor cells (e.g., defined by immunophenotyping) in the blood sample may be identified as suitable for an anti-CD70 therapy (e.g., an anti-CD70 CAR-T therapy). A patient having CD70+ cells (e.g., at least 5%, at least 10%, at least 15%, at least 20%, at least 25% or higher) in tumor cells (e.g., defined by immunophenotyping) in the blood sample may be identified as suitable for an anti-CD70 therapy (e.g., an anti-CD70 CAR-T therapy).

In some embodiments, any of the anti-CD70 antibodies disclosed herein (e.g., 11E12E8, 4E6G9, or 3H11D12E7, or a functional variant thereof, e.g., humanized antibodies thereof) may be conjugated with an imaging agent (e.g., those disclosed herein) and be used for in vivo imaging of CD70+ tumors in a human patient.

In specific examples, an anti-CD70 antibodies disclosed herein (e.g., 11E12E8, 4E6G9, or 3H11D12E7, or a functional variant thereof) may be used in an immunohistochemistry (IHC) assay for detecting and/or quantifying CD70 in a biological sample. IHC staining is a common assay method for diagnosis of target cells and/or antigens in tissue samples. An IHC assay typically would involve sample preparation, sample labeling, and target cell/antigen detection. Sample preparation is important for mainlining cell morphology, tissue architecture, and/or antigenicity of the target antigen/epitope. It may involve tissue collection, fixation, and sectioning. In some instances, the biological sample can be prepared by immersing excised tissue samples in a formaldehyde solution and then embedding the samples in paraffin wax to produce formalin-fixed and paraffin-embedded (FFPE) samples. In some embodiments, the biological samples may be treated to reduce non-specific immunostaining, for example, to block or quench endogenous biotin or enzymes that may affect staining results. For example, the samples can be incubated with a buffer that blocks the reactive sites to which the primary or secondary antibodies may otherwise bind. Common blocking buffers include normal serum, non-fat dry milk, BSA, or gelatin. The samples can then be incubated with the anti-CD70 antibody (the primary antibody) as disclosed herein (e.g., at a concentration of about 1-10 μg/ml as disclosed herein).

In some instances, the anti-CD70 antibody may be conjugated with a detectable label, e.g., those disclosed herein. In some instances, a secondary antibody that binds the anti-CD70 antibody and is conjugated with a detectable label may be used to amplify the readout signal. After washing the biological samples to remove unbound antibodies, signals released from the antibodies bound to the target antigen in the samples can be detected and analyzed via routine technology.

In some instances, a second staining after the immunohistochemical staining of the target antigen, can be performed to provide contrast that helps the primary stain stand out. For example, the second staining may show specificity for specific classes of biomolecules. Alternatively, the second staining may stain the whole cell. Both chromogenic and fluorescent dyes are available to provide a vast array of reagents to fit various experimental designs. Examples include hematoxylin, Hoechst stain and DAPI.

In some embodiments, any of the anti-CD70 antibodies disclosed herein (e.g., 11E12E8, 4E6G9, or 3H11D12E7, or a functional variant thereof) can be used to detect presence of CD70 in a sample, such as a biological sample as those described herein. In other embodiments, the anti-CD70 antibody disclosed herein may be used to quantify (measure) the level of CD70 in the sample such as the biological sample. For example, the anti-CD70 antibody can be used to measure the relative amount of CD70 in a biological sample, e.g., normalized against an internal control (e.g., expression level of a housekeeping gene or intensity of the second staining disclosed herein). In another example, the anti-CD70 antibody can be used to determine qualitative relative abundance of CD70 in biological samples.

Any patient identified by a method disclosed herein as suitable for an anti-CD70 therapy may be subject to a treatment comprising at least one anti-CD70 agent. In some examples, the anti-CD70 is an anti-CD70 antibody. In other examples, the anti-CD70 agent can be genetically engineered T cells expressing an anti-CD70 CAR (anti-CD70 CAR-T cells), for example, those disclosed in WO 2019/097305, and WO2019/215500 , the relevant disclosures of each of which are incorporated by reference for the subject matter and purpose referenced herein.

### IV. Kits for Detecting CD70 Antigen or CD70+ Cells

The present disclosure also provides kits for use in detecting or quantifying a human CD70 antigen or CD70+ cells in a sample, such as a biological sample obtained from a patient having or suspected of having a disease involving CD70+ cells, for example, a solid tumor or a hematological malignancy. Such kits can include one or more containers comprising any of the anti-CD70 antibodies disclosed herein, for example, 11G12B6, 11E12E8, 4E6G9, 3H11D12E7, 19H7E4, 18F8A8, or 16D7C8.

In some embodiments, the kit can comprise instructions for use in accordance with any of the methods described herein. The included instructions can comprise a description of detecting or quantifying the CD70 antigen or CD70+ cells in a sample as described herein. Instructions supplied in the kits of the invention are typically written instructions on a label or package insert (e.g., a paper sheet included in the kit), but machine-readable instructions (e.g., instructions carried on a magnetic or optical storage disk, or available via an internet address provided in the kit) are also acceptable.

The kits of this invention are in suitable packaging. Suitable packaging includes, but is not limited to, vials, bottles, jars, flexible packaging (e.g., sealed Mylar or plastic bags), and the like. The kits may comprise one or more aliquots of an anti-CD70 antibody described herein. Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (1RL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLE 1

### Generation of Anti-CD70 Antibodies

Immunization of mice and serum antibody titer determination were performed as described herein. BALB/c mice were immunized with recombinant human CD70 tagged with mouse IgG2a Fc (AcroBiosystems Cat#CDL-H525a) protein using either CFA or IFA as the adjuvant. Seven days after each boost, serum was separated from the blood samples, and antibody titers were determined by indirect ELISA. The coating antigens were:

A: Huma CD70-mouse IgG2a Fc

B: Mouse IgG2a protein (AcroBiosystems Cat# IGA-M5207)

The coating antigens were prepared in Phosphate Buffered Saline (PBS), pH 7.4, at 1 μg/ml and 100 μl/well. The secondary antibody was an anti-mouse IgG (FAB specific)-HRP antibody produced in goats.

Based on the antibody titers determined by indirect ELISA, one mouse was selected for cell fusion after the final boost, using a standard hybridoma protocol. After fusion and one round of subcloning, 18 hybridoma subclones representing 10 ELISA-positive clones (8F8, 9Al2, 11E12, 16D7, 16F11, 18F8, 4E6, 11G12, 19H7, 3H11D12) were subjected to ELISA screening using the same two antigens mentioned above. The results are shown in Table 2. For ten subclones (indicated in bold), the hybridomas were scaled-up for antibody sequencing and monoclonal antibody production.

TABLE 2
Positive hybridoma culture supernatants

| | | | Monoclonal isotype if |
|---|---|---|---|
| | OD 450 | | determined (Heavy |
| Cell Line | A | B | Chain, Light Chain) |
| 8F8E6 | 1.008 | 0.172 | |
| 8F8F9 | 1.103 | 0.136 | IgG1, Kappa |
| 9A12B4 | 2.016 | 0.131 | IgG1, Kappa |
| 9A12G7 | 1.841 | 0.150 | |
| 11E12C5 | 0.681 | 0.113 | |
| 11E12E8 | 1.007 | 0.107 | IgG1, Kappa |
| 16D7C8 | 2.125 | 0.154 | IgG3, Kappa |
| 16D7E8 | 2.007 | 0.144 | |
| 16F11B2 | 2.075 | 0.121 | IgG1, Kappa |
| 16F11D3 | 2.081 | 0.112 | |
| 18F8A3 | 1.763 | 0.183 | |
| 18F8A8 | 1.967 | 0.146 | IgG1, Kappa |
| 4E6G9 | 2.514 | 0.121 | IgG1, Kappa |
| 11G12B6 | 2.558 | 0.121 | IgG1, Kappa |
| 19H7E2 | 1.837 | 0.117 | |
| 19H7E4 | 1.849 | 0.134 | IgG1, Kappa |
| 3H11D12H5 | 1.728 | 0.130 | |
| 3H11D12E7 | 1.868 | 0.115 | IgG1, Kappa |
| PC(Antiserum | 2.460 | 1.911 | |
| 1:1,000) | | | |
| NC(medium) | 0.107 | 0.101 | |

PC, Positive control from the selected mouse pre-sacrifice;
NC, negative control (hybridoma medium).

### Example 2

### Evaluation of Anti-CD70 Antibodies

The ten monoclonal antibodies purified from hybridoma supernatants noted in Example 1 above were screened by manual IHC staining on a panel of CD70 positive and negative cells lines. A control antibody was also included in the screen (RnD Systems, Cat. #MAB2738). Briefly, formalin-fixed paraffin-embedded (FFPE) tissue sections were baked at 60° C. for 30 minutes, deparaffinized in xylene, rehydrated through gradually decreasing concentrations of ethanol solutions and washed with distilled water. Heat induced epitope retrieval (HIER) step was performed using the Decloaking Chamber sets at Program 4 (40 minutes at 95° C.) with 1× EDTA Decloaker solution. Tissue sections were allowed to cool to 80° C., removed from the chamber, washed with DI water and then 1× TBS-T solution. The following steps were performed at room temperature with 1× TBS-T wash cycles in between each step. Background staining was minimized by incubation with Peroxidazed 1 and protein block solutions for 5 minutes and 10 minutes, respectively. Next, primary antibody (at 10 μg/mL) was applied and incubated for 60 minutes, followed by incubation with secondary antibody for 30 minutes. The antibody-antigen binding was visualized by brown DAB stain for 10 minutes and counter-stained blue with Hematoxylin (diluted in water by 2-fold) for 5-10 seconds. Tissue slides were then thoroughly rinsed with tap water to remove excess Hematoxylin, dehydrated through gradually increased concentrations of ethanol solutions, cleared in xylene, and cover-slipped using Cytoseal™ XYL solution. Upon completion of IHC staining experiment, stained tissues were scanned using a Pannoramic MIDI II brightfield whole slide scanner (3DHISTECH Ltd.) and staining was visualized using CaseViewer software (3DHISTECH Ltd.). The results were summarized in Table 3 below.

TABLE 3
IHC staining with hybridoma supernatants of CD70 positive cell lines and CD70
negative cell line K562

| | K562 | ACHN | Nomo-1 | MM.1S | HuT78 | HH | MJ |
|---|---|---|---|---|---|---|---|
| Control | neg | 1+/2+ | 2+/3+ | neg/1+ | 1+/2+ | 2+ | 3+ |
| 11G12B6 | blush | 2+ | 3+ | 2+ | 2+ | 2+ | 3+ |
| 11E12E8 | blush | 2+/3+ | 3+ | blush/1+ | 3+ | 3+ | 3+ |
| 4E6G9 | 1+ | 2+/3+ | 3+ | 3+ | 3+ | 3+ | 3+ |
| 3H11D12E7 | 2+ | 2+ | 3+ | blush/2+ | 3+ | 3+ | 3+ |
| 19H7E4 | 2+ | 2+ | 2+ | 2+ | 2+ | 2+ | 2+ |
| 18F8A8 | 2+ | 2+ | 2+ | 2+ | 2+ | 2+ | 2+ |
| 16D7C8 | 2+ | 2+ | 2+ | 2+ | 2+ | 2+ | 2+ |
| 9A12B4 | neg | blush, P | neg | 1+ | blush | blush | blush |
| 16F11B2 | blush | blush, P | blush | 1+ | blush | blush | blush |
| 8F8F9 | neg | neg | neg | neg | neg | neg | neg |

(neg) no staining; (1+) weak positive staining; (2+) moderate positive staining; (3+) strong positive staining. Positive staining can be found at the cell membrane, membrane cytoplasmic and/or punctate cytoplasmic. Blush refers to background staining.

Antibodies that showed positive staining by IHC were sequenced from hybridoma cells using standard technologies. Their heavy chain variable region sequences and light chain variable region sequences are provided in the sequence table below.

Seven out of ten hybridoma derived antibodies exhibited similar CD70 staining patterns compared to Control Ab on CD70 positive cell lines. These antibodies were further evaluated for their binding affinity and specificity by IHC as detailed below.

The following IHC evaluation was performed using a Leica BOND™ RX autostainer and BOND™ Polymer Refine Detection kit. These antibodies were tested at 5 μg/mL or 8 μg/mL concentration on a panel of CD70 positive cell line controls and a renal clear cell carcinoma tissue microarray (RCC TMA). All incubation steps were performed at room temperature with wash cycles in between each step unless otherwise stated. Briefly, pretreatment of FFPE slides was performed using the Bake and Dewax function. It was followed by a HIER step with Epitope Retrieval solution 2 (ER2) for 20 minutes at 100° C. Next, tissues were incubated with primary antibody for 60 minutes, followed by incubation with post primary and then polymer reagents for 15 minutes each. The antibody-antigen binding was visualized by brown DAB Refine stain for 10 minutes and counter-stained blue with Hematoxylin for 5 minutes. Tissue slides were then dehydrated offline through gradually increased concentrations of ethanol solutions, cleared in xylene, and coverslipped using Cytoseal™ XYL solution. Upon completion of IHC staining experiment, stained tissues were scan using a Pannoramic MIDI II brightfield whole slide scanner and staining was visualized using CaseViewer software.

The results from this experiment were summarized in Table 4.

TABLE 4
IHC staining with purified monoclonal antibodies of CD70 positive cell lines and
CD70 negative cell line K562 using the Leica BOND platform.

| | | K562 | ACHN | Nomo-1 | MM.1S | HuT78 | HH | MI |
|---|---|---|---|---|---|---|---|---|
| 5 μg/mL | 3H11D12E7 | 2+ | 2+ | 3+ | 3+ | 3+ | 3+ | 3+ |
| 5 μg/mL | 4E6G9 | 1+ | 2+ | 3+ | 1+ | 3+ | 3+ | 3+ |
| 5 μg/mL | 16D7C8 | 3+ | 2+ | 3+ | 3+ | 2+/3+ | 3+ | 3+ |
| 5 μg/mL | 18F8A8 | 2+/3+ | 2+ | 3+ | 3+ | 3+ | 3+ | 3+ |
| 5 μg/mL | 19H7E4 | 2+ | 1+/2+ | 3+ | 2+ | 1+/2+ | 2+ | 3+ |
| 8 μg/mL | 11E12E8 | 2+ | 2+ | 3+ | 2+/3+ | 2+ | 3+ | 3+ |
| 8 μg/mL | 11G12B6 | 2+ | 2+ | 3+ | 2+/3+ | 2+/3+ | 3+ | 2+/3+ |

(neg) no staining; (1+) weak positive staining; (2+) moderate positive staining; (3+) strong positive staining. Positive staining can be found at the cell membrane, membrane cytoplasmic and/or punctate cytoplasmic.

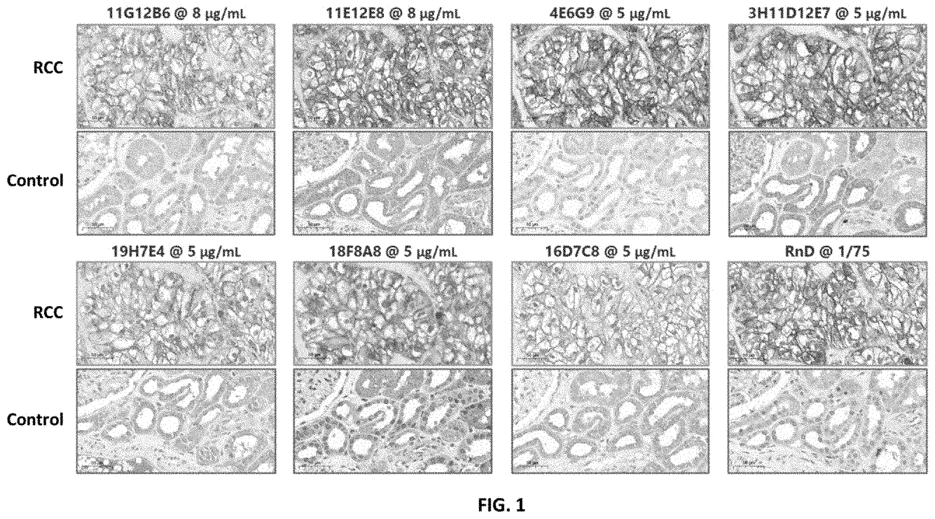

The staining on RCC tissues versus tissues adjacent to the RCC cancer tissue provided good separation of signal over noise, which indicated the specificity of the antibodies to detect CD70 antigen. As shown in FIG. 1, positive staining was observed at the cell membrane, membrane cytoplasmic and/or punctate cytoplasmic of RCC tissues with all seven antibody candidates. 4E6G9, 3H11D12E7 and 11E12E8 provided stronger staining intensity relative to the others. The antibody 4E6G9 provided strong staining of CD70 on RCC tissues while maintaining a low to negative background staining on cancer adjacent kidney tissues. Two more antibodies (3H11D12E7 and 11E12E8) also provided strong staining of CD70 on RCC tissues but with higher background staining on cancer adjacent kidney tissues compared to 4E6G9.

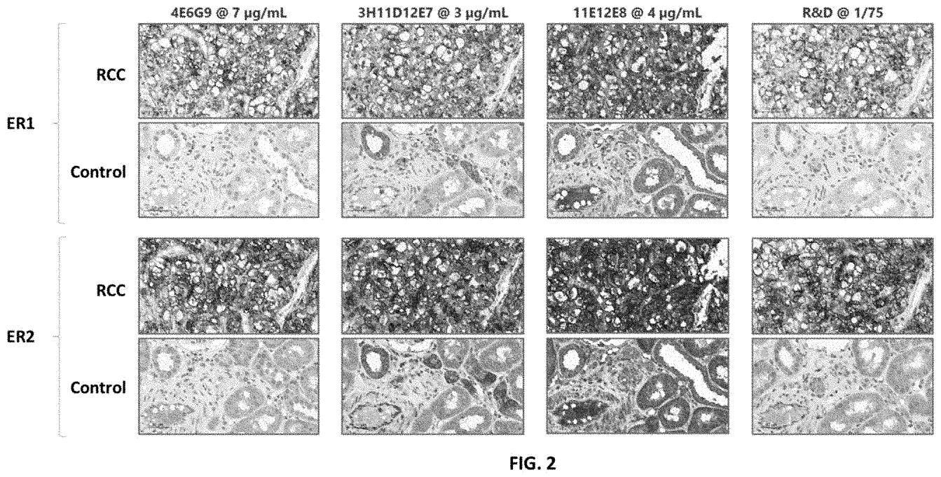

Antibodies 4E6G9, 3H11D12E7 and 11E12E8 were further evaluated by IHC using the same staining protocol but with different antigen retrieval solutions (Epitope Retrieval solution 1 and Epitope Retrieval solution 2) and antibody test concentrations. The test concentration of 4E6G9 was increased, while 3H11D12E7 and 11E12E8 were lowered. This experiment was designed to obtain strong staining signal, while maintaining a low noise level in RCC tissues and cancer adjacent kidney tissues, respectively. The data are summarized in Table 5 and FIG. 2.

TABLE 5
IHC staining with purified monoclonal antibodies of CD70 positive cell lines and
CD70 negative cell line K562 using different epitope retrieval solutions.

| | | | K562 | ACHN | Nomo-1 | MM.1S | HuT78 | HH | MJ |
|---|---|---|---|---|---|---|---|---|---|
| 4E6G9 | 7 μg/mL | ER1 | blush | 2+ | 3+ | 1+/2+ | 3+ | 2+/3+ | 2+/3+ |
| | | ER2 | 1+/2+ | 2+ | 3+ | 2+/3+ | 3+ | 3+ | 3+ |
| 11E12E8 | 4 μg/mL | ER1 | 2+ | 2+ | 3+ | 3+ | 2+/3+ | 2+/3+ | 3+ |
| | | ER2 | 2+ | 2+ | 3+ | 3+ | 2+/3+ | 2+/3+ | 2+/3+ |
| 3H11D12E7 | 3 μg/mL | ER1 | 2+ | 2+ | 3+ | 2+/3+ | 2+/3+ | 3+ | 3+ |
| | | ER2 | 2+ | 2+ | 3+ | 3+ | 2+/3+ | 2+/3+ | 2+/3+ |

(neg) no staining; (1+) weak positive staining; (2+) moderate positive staining; (3+) strong positive staining. Epitope retrieval solution 1 (ER1); Epitope retrieval solution 2 (ER2). Positive staining can be found at the cell membrane, membrane cytoplasmic and/or punctate cytoplasmic.

As shown in FIG. 2, positive staining was observed on the cell membrane, membrane cytoplasmic and/or punctate cytoplasmic of RCC tissues using 4E6G9, 3H11D12E7 or 11E12E8. With both ER1 and ER2 antigen retrieval conditions, 4E6G9 gave a strong positive CD70 staining signal in RCC tissues and the low background staining on cancer adjacent kidney tissue. Antibodies 3H11D12E7 and 11E12E8 had similar staining intensity between RCC tissues and the tubules of cancer adjacent kidney tissues.

### Example 3

### Characterization of Recombinantly Produced Anti-CD70 Antibodies

The V_{H} and V_{L} sequences of the hybridoma-produced anti-CD70 antibodies described in Examples 1 and 2 above were determined by conventional approaches and provided in Table 6 below. The heavy chain and light chain complementary determining regions (CDRs) determined by the Kabat approach are identified in boldface.

The antibodies 4E6G9 and 3H11D12E7 were chosen for recombinant expression and further development using the Leica Bond platform. The staining protocol remained the same as described in Example 2 above except that only ER2 solution was used for antigen retrieval step. The antibodies 4E6G9 and 3H11D12E7 were tested at 10 μg/mL, 5 μg/mL, 2.5 μg/mL and 1.25 μg/mL concentration. The data are summarized in Table 5.

TABLE 6
IHC staining with purified recombinantly expressed antibodies of CD70 positive
cell lines and CD70 negative cell line K562 using the Leica BOND platform.

| | | K562 | ACHN | Nomo-1 | MM.1S | HuT78 | HH | MJ |
|---|---|---|---|---|---|---|---|---|
| 4E6G9 | 10 μg/mL | blush | 1+/2+ | 3+ | blush; 2+ | 1+/2+ | 2+/3+ | 2+/3+ |
| | 5 μg/mL | blush | 1+/2+ | 3+ | blush; 1+/2+ | 1+/2+ | 2+/3+ | 2+/3+ |
| | 2.5 μg/mL | neg | 1+/2+ | 3+ | blush; 1+/2+ | 1+/2+ | 2+/3+ | 2+/3+ |
| | 1.25 μg/mL | neg | 1+/2+ | 3+ | blush; 1+ | 1+/2+ | 2+/3+ | 2+/3+ |
| 3H11D12E7 | 10 μg/mL | 1+/2+ | 1+/2+ | 3+ | 3+ | 1+/2+ | 3+ | 3+ |
| | 5 μg/mL | 1+/2+ | 1+/2+ | 3+ | 1+/2+ | 2+ | 2+/3+ | 2+/3+ |
| | 2.5 μg/mL | 1+ | 1+/2+ | 2+ | 1+/2+ | 2+ | 2+/3+ | 2+/3+ |
| | 1.25 μg/mL | blush | 1+/2+ | 2+ | 1+/2+ | 2+ | 2+/3+ | 2+/3+ |

Table 5 summarized the CD70 staining intensity observed on a panel of CD70 positive and negative cell lines. With recombinant antibody 4E6G9 there were weak to strong CD70 staining intensity in CD70 positive cell lines, while maintained a low to background staining levels with K562 cells. The background staining intensity decreased concurrently with decreased test antibody concentration of 4E6G9, while the positive CD70 staining intensity remained similar in each CD70 positive cell line across four test concentrations.

### Example 4

### Detection of CD70 Expression in Solid Tumors

IHC Solid Tumor Microarrays

CD70 expression in solid tumors is detected using the antibodies described herein to assess the prevalence of CD70 in disease. Human tissue microarrays (US Biomax, Inc.) from various solid cancer indications were evaluated for CD70 expression using a Leica BOND™ RX autostainer and BOND™ Polymer Refine Detection kit. The 4E6G9 antibody (Ms IgG1, kappa) was tested at 10 μg/mL concentration on various formalin-fixed paraffin-embedded (FFPE) human tissue microarrays. All incubation steps were performed at room temperature with wash cycles in between each step unless otherwise stated.

Briefly, pretreatment of FFPE slides was performed using the Bake and Dewax function. It was followed by a HIER step with Epitope Retrieval solution 2 (ER2) for 20 minutes at 100° C. Next, tissues were incubated with primary antibody for 60 minutes, followed by incubation with post primary and then polymer reagents for 15 minutes each. The antibody-antigen binding was visualized by brown DAB Refine stain for 10 minutes and counter-stained blue with Hematoxylin for 5 minutes. Tissue slides were then dehydrated offline through gradually increased concentrations of ethanol solutions, cleared in xylene, and coverslipped using Cytoseal™ XYL solution. Upon completion of IHC staining experiment, stained tissues were scan using a Pannoramic MIDI II brightfield whole slide scanner and staining was visualized using CaseViewer software.

As shown in Table 7 below, expression of CD70 was detected in FFPE tumor tissue samples from various solid tumors as indicated, using the anti-CD70 antibody disclosed herein (4E6G9 as an example).

TABLE 7
CD70 Expression in Solid Tumor Microarrays

| TMA | | |
|---|---|---|
| catalog # | Disease Indication | Total: CD70+ prevalence |
| GL806f | Brain glioblastoma | 22% (8 of 35 cases) |
| HN804 | Head & Neck Cancer | 13% [9 of 69 cases (primary |
| | | and metastatic squamous cell |
| | | carcinoma)] |
| | | 14% = positive fibroblasts |
| | | only (10 of 69 cases) |
| HEso- | Esophagus squamous cell | 24% (12 of 50 cases) |
| Squ127Lym- | carcinoma | |
| 01 | | |
| LC121b | Non-Small Cell Lung | overall 8% (9 of 110 cases) |
| | Cancer | 15% = Squamous Cell |
| | | Carcinoma (3 of 20 cases) |
| | | 11% = Lung Large Cell |
| | | Carcinoma (4 of 36 cases) |
| | | 3.7% = Lung Adenocarcinoma |
| | | (2 of 54 cases) |
| HLug- | Lung squamous cell | 16% (5 of 30 cases) |
| Squ090Lym- | carcinoma | |
| 01 | | |
| BCS04017b | Lung Adenocarcinoma | 4.5% (2 of 44 cases) |
| BS04116 | Lung Small Cell | 8.8% (4 of 45 cases) |
| | Carcinoma | |
| LV631 | Hepatocellular Carcinoma | 1.8% (1 of 54 cases) |
| | (HCC) | |
| LV642 | Liver | 4.7% (3 of 64 cases) |
| | hepatocholangiocarcinoma | |
| PA2082a | Pancreas carcinoma | 6.5% = Ductal adenocarcinoma |
| | | cancer cells (4 of 61 cases) |
| | | 22% = Ductal adenocarcinoma |
| | | fibroblast only (14 of 61 cases) |
| | | 20% = Adenocarcinoma cancer |
| | | cells (4 of 20 cases) |
| | | 20% = Adenocarcinoma |
| | | fibroblast only (4 of 20 cases) |
| PA1921a | Advanced stage pancreatic | 8% = cancer cells |
| | carcinoma | 14% = fibroblasts |
| HIBD- | Intrahepatic biliary cancer | 5% (5 of 100 cases) |
| Ade100PG- | | |
| 01 | | |
| ST483e | Gastric Adenocarcinoma | 7.5% (3 of 40 cases) |
| BR1401 | Breast Carcinoma | 1.5% = invasive ductal |
| | | carcinoma cancer cells (2 of |
| | | 130 cases) |
| | | 2.3% = invasive ductal |
| | | carcinoma fibroblast only (3 |
| | | of 130 cases) |
| OV802b | Ovarian carcinoma | 7% (5 of 70 cases) |
| OVC962 | Ovarian carcinoma | 5% (2 of 36 cases) |
| OS804c | Osteosarcoma | 12% (5 of 40 cases) |
| MS1001a | Malignant Mesothelioma | 20% (10 of 50 cases) |
| EMC1021 | Endometrial cancer | 1% (1 of 90 cases) |
| | | 25% (1 of 4 adenosquamous |
| | | carcinoma) |
| DT01620- | Cervical cancer | 30% (6 of 20 cases)* |
| 1007 | | |

*Source: BioIVT LLC.

Correlation between CD70 mRNA Expression and CD70 Protein Expression

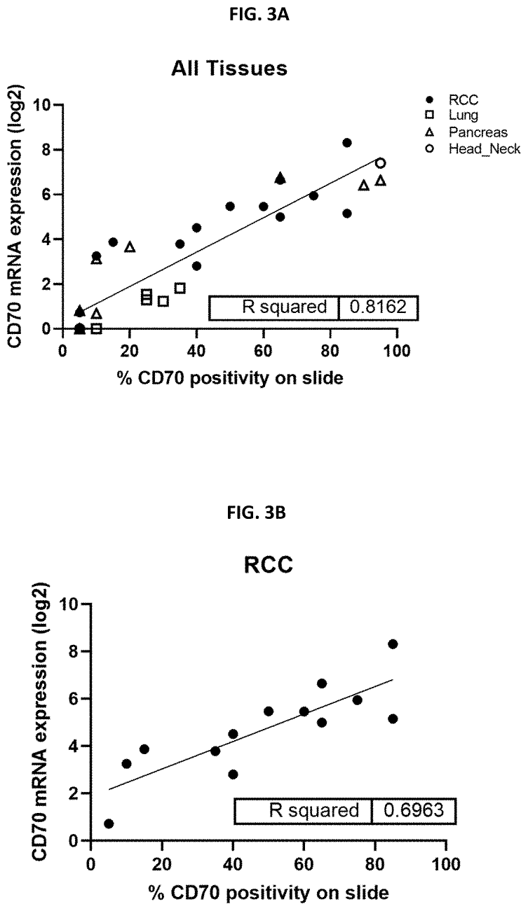

The exemplary anti-CD70 antibody disclosed above was used to detect CD70 proteins in FFPE tissue blocks from solid tumor tissues (e.g.: RCC, lung, pancreas, head & neck and glioblastoma). 45 tissue blocks were sectioned, treated and stained as described herein (see, e.g., Example 4 above). The slides were stained using the 4E6G9 described herein and scored qualitatively (% of tissue that stained positive for CD70: tumor, fibroblast, and lymphocytes). Sections (3×10 uM thickness) from the same blocks were sent to Canopy Biosciences for RNA-seq analysis. As shown in FIGS. 3A and 3B, CD70 protein expression detected by IHC staining of FFPE tissue sections correlates with mRNA levels in the same tissues. These results demonstrate that the anti-CD70 antibodies described herein are useful in assessing the expression and prevalence of CD70 in solid tumors, for example, in FFPE tumor tissue samples.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An isolated antibody, which binds a human CD70 antigen, wherein the antibody binds the same epitope of the CD70 antigen as a reference antibody or competes against the reference antibody for binding to the CD70 antigen, and wherein the reference antibody is selected from the group consisting of 11G12B6, 11E12E8, 4E6G9, 3H11D12E7, 19H7E4, 18F8A8, and 16D7C8.

2. The isolated antibody of claim 1, wherein the antibody binds the CD70 antigen expressed on a cell surface.

3. The isolated antibody of claim 1, which comprises the same heavy chain complementary determining regions and the same light chain complementary determining regions as the reference antibody.

4. The isolated antibody of claim 3, which comprises the same V_{H} and/or the same V_{L} as the reference antibody.

5. The isolated antibody of claim 1, wherein the antibody is a full-length antibody or an antigen-binding fragment thereof.

6. The isolated antibody of claim 1, wherein the antibody is a human antibody or a humanized antibody.

7. The isolated antibody of claim 1, wherein the antibody is conjugated to a detectable label.

8. A nucleic acid or a set of nucleic acids, which collectively encodes an antibody of claim 1.

9. The nucleic acid or the set of nucleic acids of claim 8, which is a vector or a set of vectors.

10. The nucleic acid or the set of nucleic acids or claim 9, wherein the vector(s) is an expression vector(s).

11. A host cell comprising the nucleic acid or the set of nucleic acids of claim 8.

12. The host cell of claim 11, wherein the host cell is a mammalian cell.

13. A method for detecting or quantifying CD70 in a sample, the method comprising:
(i) contacting an antibody of claim 1 with a sample suspected of containing CD70, and
(ii) detecting binding of the antibody to the CD70.

14. The method of claim 13, wherein the sample is suspected of containing cells expressing surface CD70.

15. The method of claim 13, wherein the sample is a biological sample from a subject.

16. The method of claim 15, wherein the biological sample is a tissue or blood sample.

17. The method of claim 13, wherein the method is an immunohistochemistry (IHC) assay.

18. The method of claim 17, wherein the sample is a formalin-fixed paraffin embedded sample.

19. The method of claim 15, wherein the subject is a human patient having or suspected of having a disease involving CD70+ cells.

20. The method of claim 15, wherein the subject is a human patient having or suspected of having a solid tumor or a hematological malignancy.

21. The method of claim 20, wherein the solid tumor or the hematological malignancy is refractory or relapsed.

22. The method of claim 20, wherein the human patient has or is suspected of having a solid tumor selected from the group consisting of pancreatic cancer, gastric cancer, ovarian cancer, cervical cancer, breast cancer, renal cancer, thyroid cancer, nasopharyngeal cancer, non-small cell lung (NSCLC), glioblastoma, and melanoma.

23. The method of claim 20, wherein the human patient has or is suspected of a hematological malignancy selected from the group consisting of peripheral T cell lymphoma (PTCL), anaplastic large cell lymphoma (ALCL), Sezary syndrome (SS), non-smoldering acute adult T cell leukemia or lymphoma (ATLL), angioimmunoblastic T cell lymphoma (AITL), and diffuse large B cell lymphoma (DLBCL).

24. The method of claim 19, wherein the biological sample comprises a tumor tissue sample, a non-tumor tissue, a sample of a tissue adjacent to a tumor site, or a combination thereof.

25. A method for identifying a human patient suitable for an anti-CD70 therapy, the method comprising:
(i) providing a biological sample from a human patient in need thereof;
(ii) contacting an antibody of claim 1 with the biological sample;
(iii) detecting binding of the antibody to CD70 in the biological sample, if any;
(iv) determining presence or measuring the level of CD70 in the biological sample based on result of step (iii); and
(v) identifying the human patient as suitable for an anti-CD70 therapy based on the presence or the level of CD70 determined in step (iv).

26. The method of claim 25, wherein the presence or the level of CD70+ cells is determined in step (iv).

27. The method of claim 25, wherein the biological sample is a tissue or blood sample.

28. The method of claim 25, wherein steps (ii)-(iii) are performed in an IHC assay format.

29. The method of claim 28, wherein the biological sample is a formalin-fixed paraffin-embedded (FFPE) sample.

30. The method of claim 25, wherein the human patient has or is suspected of having a disease involving CD70+ cells.

31. The method of claim 25, wherein the human patient has or is suspected of having a solid tumor or a hematological malignancy.

32. The method of claim 31, wherein the solid tumor or the hematological malignancy is refractory or relapsed.

33. The method of claim 31, wherein the human patient has or is suspected of having a solid tumor selected from the group consisting of pancreatic cancer, gastric cancer, ovarian cancer, cervical cancer, breast cancer, renal cancer, thyroid cancer, nasopharyngeal cancer, non-small cell lung (NSCLC), glioblastoma, and melanoma.

34. The method of claim 31, wherein the human patient has or is suspected of a T cell or B cell malignancy selected from the group consisting of peripheral T cell lymphoma (PTCL), anaplastic large cell lymphoma (ALCL), Sezary syndrome (SS), non-smoldering acute adult T cell leukemia or lymphoma (ATLL), angioimmunoblastic T cell lymphoma (AITL), and diffuse large B cell lymphoma (DLBCL).

35. The method of claim 31, wherein the biological sample comprises a tumor tissue sample, a non-tumor tissue, a sample of a tissue adjacent to a tumor site, or a combination thereof.

36. The method of claim 35, wherein the human patient is identified as suitable for the anti-CD70 therapy when the level of CD70 in the tumor tissue sample is higher than the level of CD70 in the sample of the non-tumor tissue, and/or the tissue adjacent to the tumor site.

37. The method of claim 31, wherein the human patient is identified as suitable for the anti-CD70 therapy when CD70+ disease cells are detected in the biological sample.

38. The method of claim 25, further comprising administering an anti-CD70 therapeutic agent to the human patient who is suitable to an anti-CD70 therapy identified in step (v).

39. The method of claim 38, wherein the anti-CD70 therapeutic agent is an anti-CD70 antibody or an anti-CD70 chimeric antigen receptor (CAR) T cell.

40. A method of producing an antibody binding to human CD70, the method comprising:
(i) culturing the host cell of claim 11 under conditions allowing for expression of the antibody that binds human CD70; and
(ii) harvesting the antibody thus produced from the cell culture.

41. The method of claim 38, further comprising (iii) purifying the antibody after step (ii).
